(19) United States
(12) Patent Application Publication
(10) Pub. No.: US 2023/0190261 A1
(43) Pub. Date: Jun. 22, 2023

(54) AUTOMATED LAPAROSCOPIC CLOSING METHOD AND DEVICE

(71) Applicant: BRAINCHILD SURGICAL DEVICES LLC, Brooklyn, NY (US)

(72) Inventor: Danny A. Sherwinter, Brooklyn, NY (US)

(21) Appl. No.: 17/922,470

(22) PCT Filed: Apr. 14, 2021
(86) PCT No.: PCT/US21/27203

§ 371 Date: Oct. 31, 2022

Related U.S. Application Data
(60) Provisional application No. 63/018,613, filed on May 1, 2020.

(51) Int. Cl. A61B 17/04; A61B 17/062; A61B 17/06

(52) U.S. Cl. CPC A61B 17/0469; A61B 17/0625; A61B 17/06004; A61B 17/06114; A61B 2017/00663

(57) ABSTRACT

Techniques are described for closure of a defect in material, such as closure of a laparoscopic surgical defect. A defect closure device can be inserted into the defect to capture first material adjacent to the defect. The device houses at least first and second suture pins coupled together by a suture. A first interaction with a trigger structure can force the first suture pin through the captured first material and into a containment sub-assembly. After rotating the device to capture second material adjacent to the defect, a second interaction with the trigger structure can force the second suture pin through the captured second material and into the containment sub-assembly. As such, the suture is passed through the first and second portions of the material, and the ends of the suture can be cinched, and cut to form a stitch.

### CROSS REFERENCE TO RELATED APPLICATION

This application claims the benefit of priority of U.S. Provisional Application No. 63/018,613, filed May 1, 2020, which is incorporated herein by reference in its entirety for all purposes.

### FIELD

The invention relates generally to surgical implements and methods. More particularly, embodiments relate to automated laparoscopic closure devices and defect closure methods utilizing such laparoscopic closure devices.

### BACKGROUND

With minimally invasive surgical procedures, incisions are made in the skin, subcutaneous fat, fascia, and muscle tissue. Instruments may then be introduced through these incisions to perform surgery. An example of one such surgery is laparoscopy, which is a type of minimally invasive abdominal surgery where a fiber-optic instrument is inserted through the abdominal wall to view the organs in the abdomen or to permit a surgical procedure. The incisions may be closed by sutures at the conclusion of the procedure.

Conventional techniques used to close these incisions involve use of curved needles. Some techniques involve placing a suture through the fascia, then grasping the suture within a cavity. Such techniques often rely on costly specialized equipment. Even with such specialized tools, surgeons typically rely on a camera and/or their own sense of feel, which poses a risk of injury to bowels, blood vessels, or other intra-abdominal organs. Small mistakes in positioning and/or use of such instruments can lead to sepsis and/or hemorrhage. The current methods require a learning curve for a practitioner to become proficient in these techniques.

### BRIEF SUMMARY

Embodiments provide devices and methods for closure of a defect in material, such as closure of a laparoscopic surgical defect. A defect closure device can be inserted into the defect to capture first material adjacent to the defect. The device houses at least first and second suture pins coupled together by a suture. A first interaction with a trigger structure can force the first suture pin through the captured first material and into a containment sub-assembly. After rotating the device to capture second material adjacent to the defect, a second interaction with the trigger structure can force the second suture pin through the captured second material and into the containment sub-assembly. As such, the suture is passed through the first and second portions of the material, and the ends of the suture can be cinched, and cut to form a stitch.

According to one set of embodiments, a method is provided for closure of a defect in material. The method includes: inserting an automated defect closure device into a defect in a material until a first portion of the material disposed on a first side of the defect is captured in a material capture region of the defect closure device, the defect closure device having an automated fire and release (AFR) assembly extending at least partially outside the defect, and first and second suture pins coupled together by a suture, the first suture pin being chambered in a load location; first actuating a trigger structure of the AFR assembly to force the first suture pin to pass from the load location on a first side of the material capture region, through the first portion of the material captured in the material capture region, and into a containment sub-assembly of the defect closure device on an opposite side of the material capture region from the load location, such that a first end of the suture coupled with the first suture pin is passed through the first portion of the material; repositioning the defect closure device within the defect to capture, in the material capture region, a second portion of the material disposed on a second side of the defect is captured of the defect closure device, the second suture pin being chambered in the load location subsequent to the first actuating; second actuating the trigger structure to force the second suture pin to pass from the load location, through the second portion of the material captured in the material capture region, and into the containment sub-assembly, such that a second end of the suture coupled with the second suture pin is passed through the second portion of the material; and removing the defect closure device from the defect, such that the suture is passed through the first and second portions of the material, and the first and second ends of the suture are outside the defect.

According to another set of embodiments, automated defect closure device is provided. The device includes a defect insertion region configured to be inserted into a defect in a material; a material capture region configured to capture a portion of the material adjacent to the defect when the defect insertion region is inserted into the defect; an automated fire and release (AFR) region to house a AFR assembly, wherein the material capture region is disposed between the defect insertion region and the AFR region; an automated suture cartridge (ASC) assembly to house a first suture pin and a second suture pin, each coupled to a respective end of a suture; and a containment sub-assembly. The AFR assembly is configured, upon activation, to force one of the suture pins to pass from a load location of the ASC assembly through the portion of the material captured in the material capture region and into the containment sub-assembly, such that the respective end of the suture coupled with the one of the suture pins is passed through the portion of the material.

### BRIEF DESCRIPTION OF THE DRAWINGS

The accompanying drawings, referred to herein and constituting a part hereof, illustrate embodiments of the disclosure. The drawings together with the description serve to explain the principles of the invention.

FIGS. 1A and 1B show a side view and a side cutaway view, respectively, of an illustrative automated laparoscopic closure device, according to embodiments described herein.

FIGS. 2A-2F show exemplary usage of the automated laparoscopic closure device in context of a defect in material.

FIG. 3A shows a cutaway side view of the automated laparoscopic closure device of FIGS. 1A and 1B without all of the internal components.

FIG. 3B shows a partially exploded perspective view of the automated laparoscopic closure device of FIGS. 1A and 1B illustrating one implementation for constructing the handle portion and the body portion of the housing.

FIGS. 3C and 3D show partially exploded front and top views, respectively, of the automated laparoscopic closure device of FIGS. 1A and 1B illustrating a two-piece implementation to form a unitary housing including the handle portion and the body portion.

FIGS. 3E and 3F partial side cutaway and partial perspective cutaway views, respectively, of the automated laparoscopic closure device of FIGS. 1A and 1B illustrating an implementation of the pin capture structure in context of the pin container structure and the material capture region.

FIG. 4 shows a cutaway view of an alternative implementation of an automated laparoscopic closure device that includes a suture pin cartridge, according to embodiments described herein.

FIG. 5 shows a front view of another embodiment of an automated laparoscopic closure device having a two-sided material capture region.

FIG. 6 shows an example of a pair of suture pins coupled together by a suture to illustrate features of various embodiments described herein.

FIG. 7A shows an example of the automated laparoscopic closure device in context of a defect in material in a position similar to that of FIG. 2E, but with a self-locking suture configuration.

FIG. 7B shows an example of the self-locking suture having been cut away from the automated laparoscopic closure device and having been partially cinched to close the defect in the material.

FIG. 8 shows a flow diagram of an illustrative method for closure of a defect in material, according to various embodiments described herein.

In the appended figures, similar components and/or features can have the same reference label. Further, various components of the same type can be distinguished by following the reference label by a second label that distinguishes among the similar components. If only the first reference label is used in the specification, the description is applicable to any one of the similar components having the same first reference label irrespective of the second reference label.

### DETAILED DESCRIPTION

In the following description, numerous specific details are provided for a thorough understanding of the present invention. However, it should be appreciated by those of skill in the art that the present invention may be realized without one or more of these details. In other examples, features and techniques known in the art will not be described for purposes of brevity.

Embodiments herein describe devices, and methods for using such devices, to close up a defect in a material using a suture. Embodiments are described specifically as a surgical implement for use in laparoscopic surgery. For example, in the laparoscopic surgery context, the term “material” typically includes skin, subcutaneous fat, fascia, and muscle tissue. The “defect” is a hole or incision made through the material during a surgical procedure, and the “suture” includes a strand, fiber, or other suitable material with which to for a stitch (or multiple stitches) that hold together the sides of the defect in the material. For example, in a laparoscopic procedure, a defect can provide a port for insertion of a trocar, which can be used for various purposes, including draining fluid and introducing laparoscopic hand instruments, cameras, etc. These defects can be closed with sutures at the conclusion of a procedure to prevent herniation and other complications at these sites. In some embodiments, the sutures described herein (e.g., including the self-locking sutures described herein) are absorbable or dissolvable sutures. For example, the sutures are made of a material that is dissolved, or otherwise eradicated, by inflammatory responses of the immune system; that disintegrate over time; and/or that are readily absorbed over time into the sutured material (e.g., tissue, etc.). While some descriptions herein refer to use of the sutures to “close” a defect, or the like, it will be understood that the sutures themselves may only partially close the defect. For example, devices described herein can be used to form one or more stitches across a portion of a defect using one or more sutures, and the defect can be further closed using staples, further suturing, and/or other materials.

Some conventional techniques used to close tissue defects (or, alternatively, to close any hole where one has ready and/or direct access only to one side thereof) involve use of curved needles. Some techniques involve placing a suture through the fascia, then grasping the suture extending freely in the air within a cavity (e.g., under camera guidance). This task can be extremely difficult even in experienced hands. Further, such techniques often rely on costly specialized equipment for each procedure which is unacceptable to hospitals and surgery centers.

Even with such specialized tools, surgeons typically still must rely solely on feeling their way through a cavity, and/or looking in a camera, while at the same time, risking injury to bowels, blood vessels, or other intra-abdominal organs. Small mistakes in positioning and/or use of instruments can lead to sepsis, hemorrhage, and even death. The current methods are cumbersome and require a significant learning curve for a practitioner to become proficient in the techniques.

Embodiments described herein include devices and methods for quickly, safely, and efficiently closing defects in material by suturing. Embodiments include an automated fire and release (AFR) assembly, an automated suture cartridge (ASC) assembly, and a housing assembly. The housing assembly includes a handle, a defect insertion region, and a material capture region. To close a defect in material, the defect insertion region can be pushed through the defect in the material and oriented so that the material on a first side of the defect is captured in the material capture region. The AFR assembly can be activated to engage with the ASC assembly to inject a first suture pin through the captured portion of the material on the first side of the defect and into a containment sub-assembly of the housing assembly. The first suture pin is coupled with a second suture pin (still in the ASC assembly) by a suture (e.g., natural or synthetic thread or wire). The device can be rotated to a second side of the defect (e.g., opposite the first side), such that the material on the second side of the defect is captured in the material capture region of the device. The AFR assembly can be activated again to engage with the ASC assembly to inject the second suture pin through the captured portion of the material on the second side of the defect and into the containment sub-assembly of the housing assembly. The device can be withdrawn from the defect, forming a “U” stitch is that begins at the first suture pin, passes through the material of the first then second sides of the defect, and ends at the second suture pin. The U stitch can be used to draw the sides of the defect together, and the two ends of the suture can be cut and tied to complete the stitch and hold the defect closed.

As described herein, some embodiments further include a novel type of suture sub-assembly designed to be self-closing. For example, the suture is designed with spaced engagement structures, the first suture pin includes a ratchet structure that is designed to couple with the suture and to permit one-directional passage of the spaced engagement structures of the suture. The second suture pin is designed so that injection of the second suture pin causes the second suture pin to pass through the ratchet structure and to breakaway the ratchet structure from the first suture pin. In such embodiments, withdrawing the device after injecting the second suture pin can cause the second suture pin to sequentially pull the engagement structures of the suture through the ratcheting structure, thereby self-closing the stitch.

Embodiments of the laparoscopic closure devices described herein are shown as a unitary, self-enclosed device. Some such embodiments are manufactured and distributed as disposable devices. For example, the device is packaged for use during a surgical procedure, and fully disposed of after use. Some implementations can be packaged with one or more separate components. For example, one or more suture pin cartridges are preloaded with suture pins and packaged separately from the laparoscopic closure device (e.g., in the same, or separate physical packaging), and a suture pin cartridge is installed into the laparoscopic closure device prior to use. Some such implementations are designed, so that after use of the laparoscopic closure device, the laparoscopic closure device and the suture pin cartridge installed therein can be disposed. Some such implementations can permit limited reuse of certain components. For example, the suture pin cartridges can be designed to be removed from the laparoscopic closure device after use (e.g., and prior to disposing of the laparoscopic closure device), sterilized (e.g., by placing in an autoclave, or the like), reloaded with suture pins, and repackaged for subsequent use. Other such implementations can permit different types of suture pin cartridges to be selected for use in the laparoscopic closure device. For example, different types of suture pin cartridges can support different types of suture pins (e.g., optimized to be pushed through particular types of material), different types of sutures (e.g., absorbable sutures, thicker or thinner sutures, self-locking sutures, etc.), and/or for other purposes. Other such embodiments are manufactured to permit limited replenishing and/or refurbishing. For example, embodiments can permit installation of a ASC assembly, to refill the ASC assembly with suture pins, etc., such as for suturing during a single surgical procedure on a single patient. Other embodiments can be designed for longer-term and/or more repeated use. For example, the structural assembly can be designed to permit simple removal and replacement of the ASC assembly, to reload the ASC with cartridge pins, to remove any consumed cartridge pins and/or other materials from containment sub-assembly, to refurbish or repair the AFR assembly, to sterilize portions of the AFR assembly and/or the structural assembly, etc.

The term “automated,” as used herein, is generally intended to indicate that the device is not fully manual. As such, some embodiments are described as “automated,” even though operation of those devices involves manual interaction with the device. For example, in a surgical context, conventional devices and techniques may involve a surgeon manually threading a needle with a suture, inserting the needle and suture, etc. The devices and methods described herein are at least partially automated, such as by forcing suture pins through material in a manner that automatically threads the suture through locations in the material. Some devices and method support further automation, such as by automatically advancing a next suture pin into a load position after each previous suture pin is injected through the material, by automatically returning any triggering and/or deployment structures to a reset position after actuation, etc.

Though embodiments are described in context of laparoscopic surgery, devices and methods described herein can be tailored for other (e.g., surgical or non-surgical) applications in which it is desirable to close a similar type of defect in a similar type of material using a suturing-type of technique. For example, a hole can develop in upholstery, such that the hole is through one or more layers of material. In such a case, the material may include one or more of outer fabric cushioning or padding material, and inner fabric, and devices and methods described herein can be adapted to form stitches by which to close up the defect (hole) in the material using thread or other stitching material.

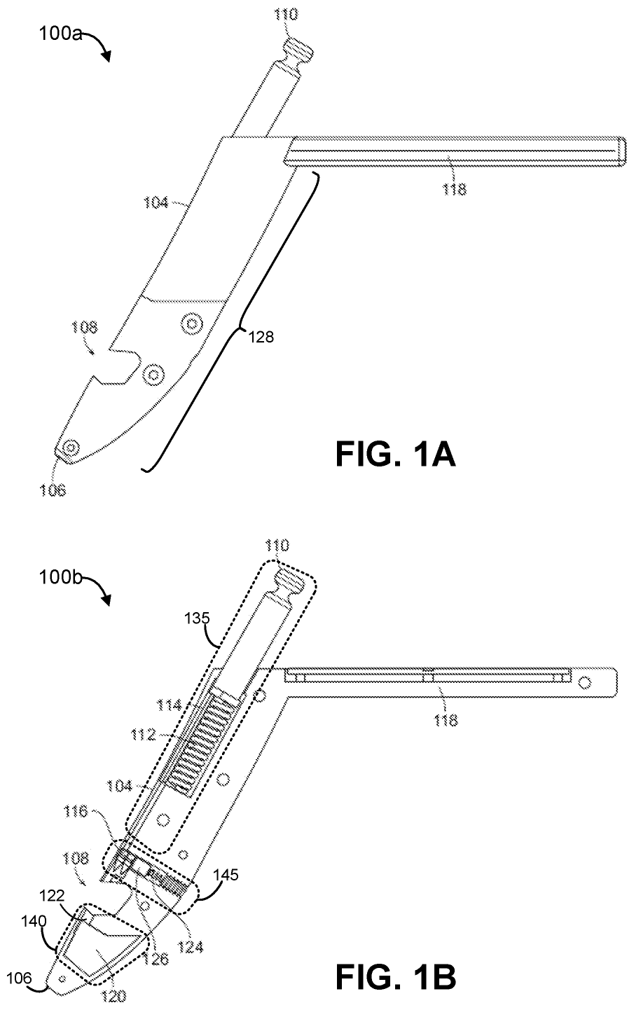

FIGS. 1A and 1B show a side view and a side cutaway view, respectively, of an illustrative automated laparoscopic closure device 100, according to embodiments described herein. FIGS. 1A and 1B include common reference designators for common components, and the two Figures are described concurrently. The automated laparoscopic closure device 100 includes a handle portion 118 and a body portion 128. In some embodiments, the handle portion 118 and the body portion 128 are coupled together in a relative orientation that forms an obtuse inner angle. Such an orientation can improve the ergonomics of the device and can make it easier to insert the automated laparoscopic closure device 100 into a defect in material. The body portion 128 can include an automated fire and release (AFR) region 104, a defect insertion region 106, and a material capture region 108 situated between the AFR region 104 and the defect insertion region 106. For added clarity, the handle portion 118 can be said to couple with the body portion 128 substantially in a proximal region of the body portion 128, and the body portion 128 can extend from there to a distal region of the body portion 128. For example, the AFR region 104 generally forms the proximal region of the body portion 128, the defect insertion region 106 generally forms the distal region of the body portion 128, and the material capture region 108 is between the proximal and distal regions.

Embodiments of the AFR region 104 are configured to structurally support components and functionality of an AFR assembly 135. The AFR assembly 135 can include a trigger structure 110, a pin deployment structure 114, and a release structure 112. As described more fully below, the trigger structure 110, pin deployment structure 114, and release structure 112 are all in structural communication, such that interacting with the trigger structure 110 causes the pin deployment structure 114 to fire, and the release structure 112 causes the pin deployment structure 114 to release. In the illustrated embodiment, the AFR region 104 is configured to fully house and support the release structure 112, to fully house and support (e.g., and help guide) the pin deployment structure 114 (at least in its released position), and to partially house the trigger structure 110. For example, as shown, the trigger structure 110 is implemented as a plunger with at least a portion of the plunger protruding from the AFR region 104 of the body portion 128 to facilitate manual interaction with the trigger structure 110. In some embodiments, the trigger structure 110 (and/or other components of the AFR assembly 135) can include a locking mechanism to hinder or prevent inadvertent firing of the trigger structure 110 (e.g., during shipping, when not in use, etc.). For example, a small switch or other structural feature can interface with the trigger structure 110 to prevent it from being inadvertently depressed.

Though various embodiments of the trigger structure 110 are illustrated and described as a plunger-type of mechanism that engages with the pin deployment structure 114, other embodiments can be implemented with any suitable means for translating a triggering action to a pin deployment action. In some embodiments, the trigger structure 110 includes a structure that extends into the handle portion 118, such as an elongated jaw-type structure similar to a handle of locking pliers, a curved trigger similar to that of a pistol, etc.; and the trigger structure 110 is engaged by squeezing the components against the handle portion 118 of the automated laparoscopic closure device 100 to engage the pin deployment structure 114. In other embodiments, the handle portion 118 itself can be configured as the trigger structure 110. For example, the handle portion 118 is coupled with the body portion 128 by a hinge mechanism that is coupled with the pin deployment structure 114 (e.g., via gears, cams, etc.), and moving the handle portion 118 via the hinge mechanism triggers engagement of the pin deployment structure 114. Further, while the pin deployment structure 114 is shown as a single rod coupled directly to the trigger structure 110, the pin deployment structure 114 can include any suitable means for coupling with the trigger structure 110 and/or for engaging the suture pins 116. For example, the pin deployment structure 114 can be coupled with the trigger structure 110 using one or more gears, cams, levers, springs, etc. Such implementations can translate the types of motion and/or force used to actuate the trigger structure 110 into different motion and/or force of the pin deployment structure 114. For example, a relatively small and low-force squeezing motion applied to a trigger structure 110 in the handle portion 118 can be translated by various components (e.g., lever, gears, etc.) into a larger and higher-force linear motion of the pin deployment structure 114 in a pin deployment direction (i.e., in the direction for pushing a suture pin through material, as described herein).

Embodiments of the defect insertion region 106 can be shaped to facilitate smooth, safe insertion into the defect. For example, the defect insertion region 106 can have a generally smooth, tapered shape, so that the defect insertion region 106 smoothly pushes past the material around the defect. The defect insertion region 106 can also have a dull and/or rounded end to mitigate any damage to features inside the defect while the device is being inserted, such as damage to internal organs, etc. In some embodiments, as described more fully below, the defect insertion region 106 can include structures to support a containment sub-assembly 140 to capture and contain suture pins 116 after they are deployed. For example, the containment sub-assembly 140 can include a pin capture structure 122 and a pin container structure 120.

Embodiments of the material capture region 108 can be shaped as a notch, or other suitable cutout, in the body portion 128. During use of the automated laparoscopic closure device 100, material (e.g., skin, fascia, subcutaneous fat, etc.) on a side of the defect is captured in the material capture region 108. While the material is captured there, the AFR assembly 135 is used to fire a suture pin 116 through the captured material. For example, the AFR assembly 135 is configured, so that the trigger structure 110 can be used to engage the pin deployment structure 114 with a suture pin 116 and to push the suture pin (e.g., or pull the suture pin 116 in other implementations), along with a suture coupled thereto, through the captured material.

Embodiments of the automated laparoscopic closure device 100 further include an automated suture cartridge (ASC) assembly 145. In the illustrated embodiment, the ASC assembly 145 is disposed “above” the material capture region 108, between the AFR assembly 135 and the material capture region 108 (e.g., in the AFR region 104). As described below, in other embodiments, the ASC assembly 145 can be disposed “below” the material capture region 108, in the defect insertion region 106. The ASC assembly 145 can include multiple suture pins 116, and one or more suture pin advancement structures. The illustrated implementation only supports a single pair of suture pins 116, and the suture pins 116 are held in a chamber, with the first suture pin 116 of the pair in a load position. As described herein, other implementations can support larger numbers of suture pins 116, such as by using suture pin cartridges. Though not explicitly shown, the pair of suture pins 116 are coupled together by a suture. The illustrated advancement structures include a block 126 and spring 124 arranged to apply lateral pressure on the suture pins 116.

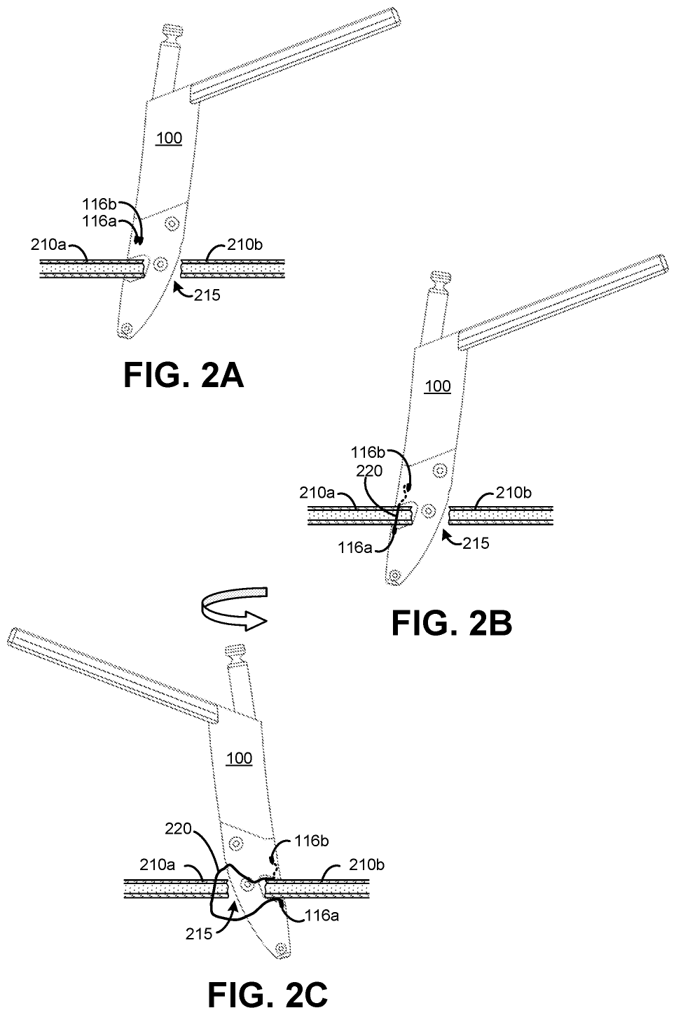

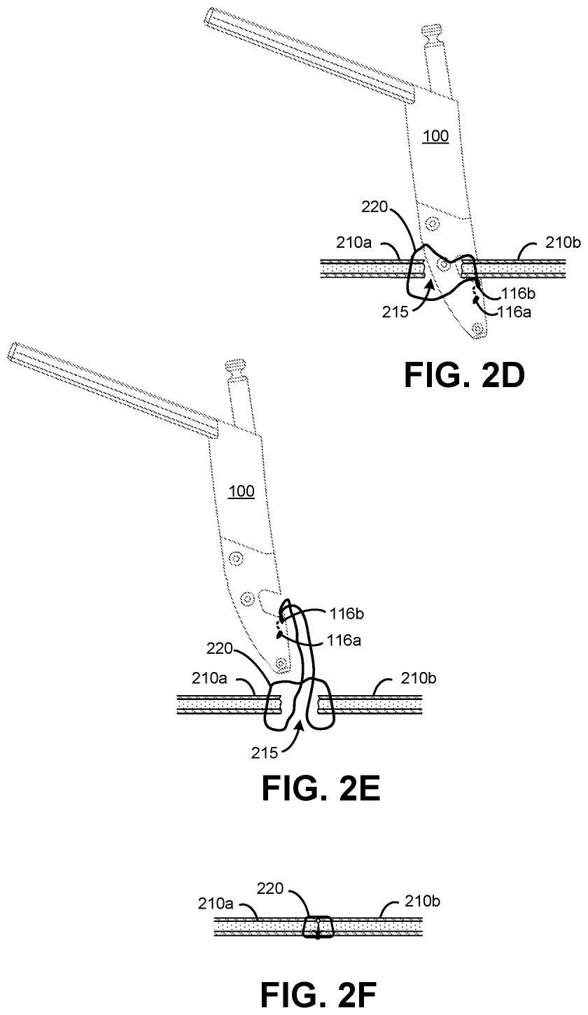

For the sake of illustration, FIGS. 2A-2F show exemplary usage of the automated laparoscopic closure device 100 in context of a defect 215 in material 210. As illustrated in FIG. 2A, the automated laparoscopic closure device 100 is inserted into a defect 215 in material 210, such that first material 210a on a first side of the defect 215 is captured in the material capture region 108 of the automated laparoscopic closure device 100. A pair of suture pins 116 is shown for clarity, though the suture pins 116 are chambered within the automated laparoscopic closure device 100 and would not be visible. The first suture pin 116a is in the load position in the chamber. Operation of the automated laparoscopic closure device 100 in the position of FIG. 2A can involve depressing the trigger structure 110 to cause the pin deployment structure 114 (e.g., illustrated in FIG. 1B as a firing rod) to travel in the direction of, and to engage with, the first suture pin 116a. Some embodiments are configured for manual use, such as by a surgeon. For example, depressing the trigger structure 110 can involve the surgeon pressing down on a plunger-like structure of the trigger structure 110. Other embodiments are configured for further automation, such as by coupling the trigger structure 110 with a machine configured to depress the trigger structure 110, as instructed.

Further depressing the trigger structure 110 causes the pin deployment structure 114 to push the first suture pin 116 from its chambered position, through the first material 210a (captured in the material capture region 108), and into the pin capture structure 122 of the containment sub-assembly 140. For example, FIG. 2B shows the first suture pin 116a having been pushed through the first material 210a and into the pin capture structure 122, thereby pushing a connected portion of a suture 220 through the first material 210a. Embodiments of the pin deployment structure 114 and/or the suture pin 116 are configured, so that the pin deployment structure 114 removably couples with the suture pin 116. For example, the pin deployment structure 114 includes a rod, or other structure, that friction fits (e.g., or magnetically couples, etc.) with a similarly sized depression, notch, or other feature of the suture pin 116. As such, while the pin deployment structure 114 is pushing the suture pin 116 through the material 210, the pin deployment structure 114 is also holding on to the suture pin 116 securely enough to avoid the suture pin 116 decoupling from the pin deployment structure 114 within the material 210, or on the opposite side of the defect 215. In such embodiments, the pin capture structure 122 is configured to decouple the suture pin 116 from the pin deployment structure 114 after the suture pin 116 has been pushed through the material 210. In effect, the pin deployment structure 114 includes any suitable means to hold on to the suture pin 116 during deployment, and the pin capture structure 122 includes any suitable means to hold on to the suture pin 116 more tightly than does the pin deployment structure 114, thereby pulling (e.g., dislodging, pulling, grabbing, etc.) the suture pin 116 from the pin deployment structure 114. It can also be seen in FIG. 2B that, after deployment of the first suture pin 116a through the first material 210a, the other end of the suture is coupled with the second suture pin 116b, which is still in the chamber of the automated laparoscopic closure device 100.

The release structure 112 (e.g., illustrated in FIG. 1B as a spring) can cause the pin deployment structure 114 to disengage from the first suture pin 116 and to return to a reset position. For example, releasing pressure on the trigger structure 110 causes the trigger structure 110 to return to the reset position under return pressure from the release structure 112. With the first suture pin 116a and the pin deployment structure 114 out of the way, the lateral pressure from the advancement structures (i.e., the block 126 and spring 124) can cause the second suture pin 116b to be pushed into the load position in the chamber. The automated laparoscopic closure device 100 can be repositioned (e.g., rotated) without being removed from the defect so as to capture second material 210b around the defect 215 in the material capture region 108 (e.g., across the defect 215 from the first material 210a). For example, FIG. 2C shows the automated laparoscopic closure device 100 turned approximately 180 degrees to capture the second material 210b in the material capture region 108. FIG. 2C also shows that the suture 220 is still running through the first material 210a, with one end of the suture 220 coupled with the first suture pin 116a in the pin capture structure 122, and the other end of the suture 220 coupled with the second suture pin 116b now in the load position of the chamber.

While in the configuration of FIG. 2C, depressing the trigger structure 110 a second time causes the pin deployment structure 114 to travel in the direction of, and to engage with, the second of the suture pins 116b. Further depressing the trigger structure 110 pushes the second suture pin 116b from its chambered position, through the second material 110b now captured in the material capture region 108, and into the pin capture structure 122 of the containment sub-assembly 140. Pushing the second suture pin 116b into the pin capture structure 122 can cause the first suture pin 116a to be further pushed into the pin container structure 120. For example, FIG. 2D shows the second suture pin 116b having been pushed through the second material 210b and into the pin capture structure 122, thereby pushing a connected portion of a suture 220 through the second material 210b and pushing the first suture pin 116a into the pin container structure 120 (inside the automated laparoscopic closure device 100).

Because the two suture pins 116 are connected by the suture 220, and the suture pins 116 are both being held by the containment sub-assembly 140 (the first in the pin container structure 120, and the second in the pin capture structure 122), removing the automated laparoscopic closure device 100 from the defect effectively leaves in place a stitch between the first and second material 210. For example, FIG. 2E shows the automated laparoscopic closure device 100 removed from the defect 215 with the two suture pins 116 held by the containment sub-assembly 140. It can clearly be seen in FIG. 2E that the suture 220 begins inside the pin container structure 120 (coupled with the first suture pin 116a), travels up through the first material 210a, travels down through the second material 210b, and ends in the pin capture structure 122 (coupled with the second suture pin 116b). Pulling the ends of the suture 220 more tautly can pull together the first and second material 210, thereby closing up the defect 215. The ends of the suture 220 can then be cut and tied to complete the stitch. For example, FIG. 2F shows that the suture 220 has been pulled taut, and the defect 215 has been pulled closed. If can be seen in FIG. 2F that the ends of the suture 215 have been cut and tied to complete the stitch.

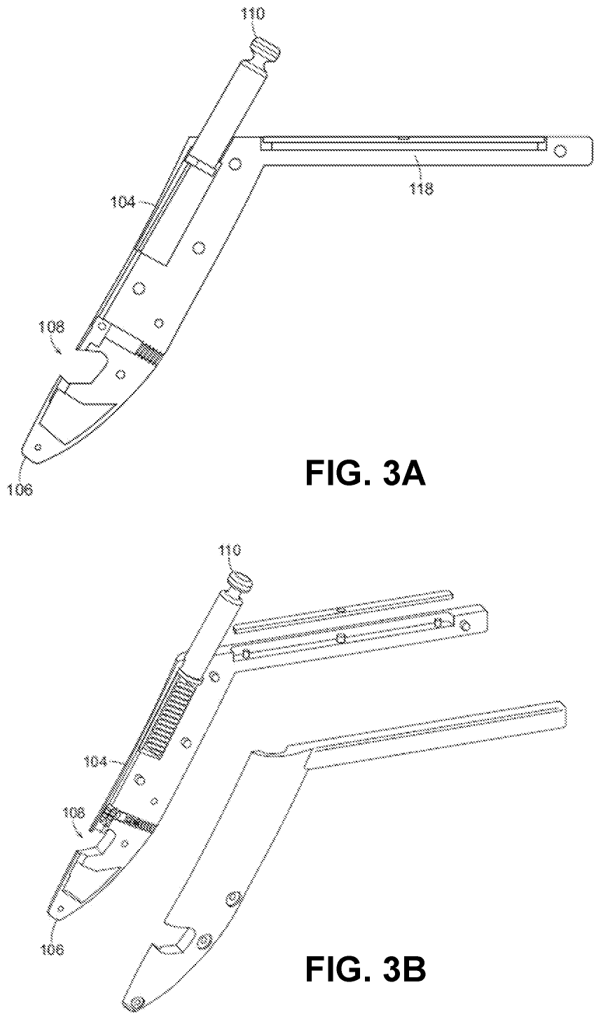

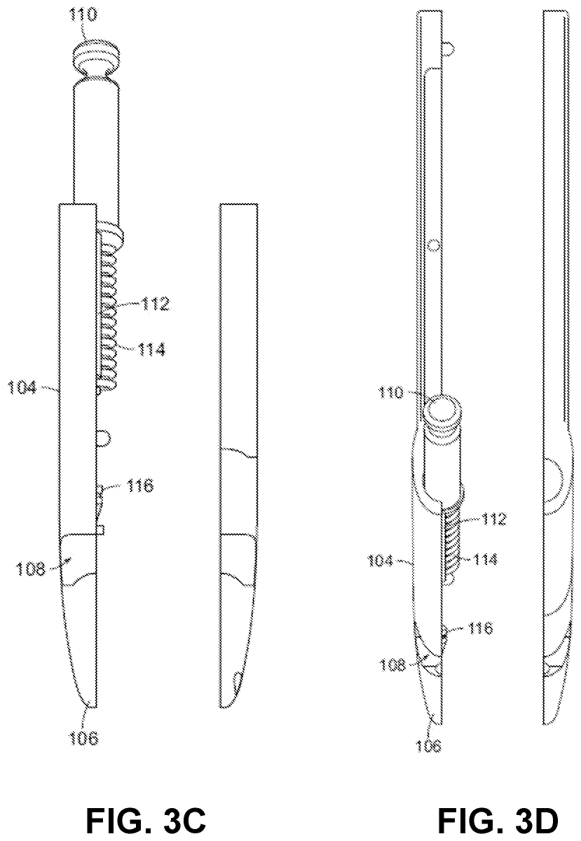

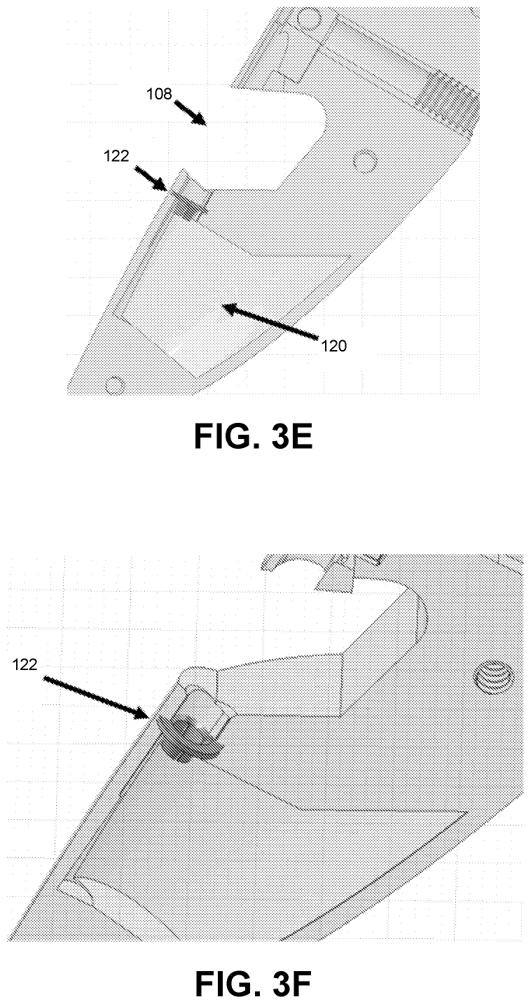

For added clarity, FIGS. 3A-3E show additional views of embodiments of automated laparoscopic closure devices. FIG. 3A shows a cutaway side view of the automated laparoscopic closure device 100 of FIGS. 1A and 1B without all of the internal components. For example, FIG. 3A does not show the release structure 112, the ASC assembly 145, the suture pins 116, and other components. FIG. 3B shows a partially exploded perspective view of the automated laparoscopic closure device 100 of FIGS. 1A and 1B illustrating one implementation for constructing the handle portion 118 and the body portion 128 of the housing. FIGS. 3C and 3D show partially exploded front and top views, respectively, of the automated laparoscopic closure device 100 of FIGS. 1A and 1B illustrating a two-piece implementation to form a unitary housing including the handle portion 118 and the body portion 128. FIGS. 3E and 3F partial side cutaway and partial perspective cutaway views, respectively, of the automated laparoscopic closure device 100 of FIGS. 1A and 1B illustrating an implementation of the pin capture structure 122 in context of the pin container structure 120 and the material capture region 108.

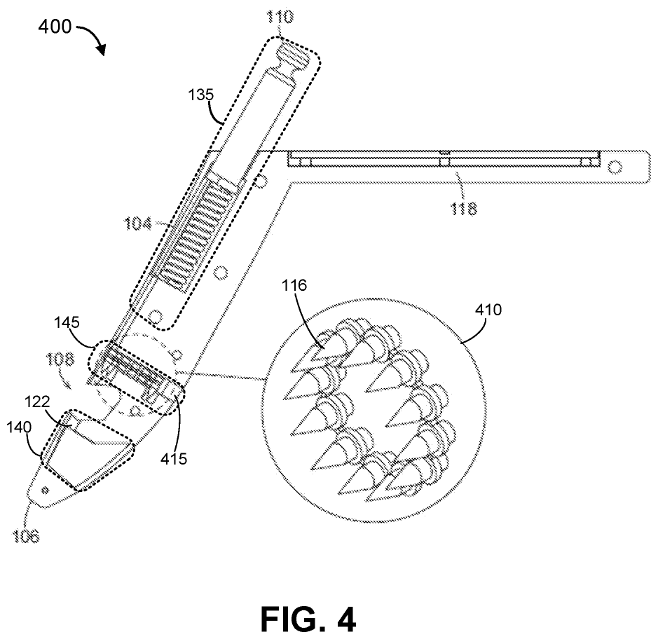

FIG. 4 shows a cutaway view of an alternative implementation of an automated laparoscopic closure device 400 that includes a suture pin cartridge 410, according to embodiments described herein. Some implementations of the device 400 are implemented in a form factor similar to that shown in FIGS. 1A-3D. Other implementations can modify the form factor in any suitable manner. As in FIGS. 1A and 1B, the automated laparoscopic closure device 400 includes a handle portion 118 and a body portion 128. The body portion 128 can include an AFR region 104, a defect insertion region 106, and a material capture region 108 situated between the AFR region 104 and the defect insertion region 106. Embodiments of the AFR region 104 are configured to structurally support components and functionality of an AFR assembly 135, which can include a trigger structure 110, a pin deployment structure 114, and a release structure 112. Some embodiments of the defect insertion region 106 include structures to support a containment sub-assembly 140 to capture and contain suture pins 116 after they are deployed, and embodiments of the containment sub-assembly 140 can include a pin capture structure 122 and a pin container structure 120.

Embodiments of the automated laparoscopic closure device 100 further include an automated suture cartridge (ASC) assembly 145 that supports one or more suture pin cartridges 410. The illustrated embodiment shows a single suture pin cartridge 410 configured to hold twelve suture pins 116. As described above, the suture pins 116 can be configured as pairs, wherein each pair is coupled together by a respective suture 220 (not shown). For example, the illustrated suture pin cartridge 410 can hold six suture pin 116 pairs to form six stitches with six respective sutures 120.

The suture pin cartridge 410 can be advanced by any suitable suture pin advancement structures 415. Some suture pin advancement structures 415 automate the advancement of the suture pin cartridge 410. In one implementation, though not explicitly shown, the suture pin advancement structures 415 includes a pawl and ratchet under tension by a tension spring and coupled with the pin deployment structure 114 (or otherwise with the trigger structure 110). Interaction with the trigger structure 110 (e.g., pressing down on the plunger mechanism) engages the pawl and ratchet assembly and causes the suture pin cartridge 410 to rotate, thereby advancing a next suture pin 116 to a load position in the chamber. Such a pawl and ratchet assembly can be located adjacent to the suture pin cartridge 410, central to the suture pin cartridge 410 (e.g., such that the ratchet mechanism is substantially at a rotational axis of the suture pin cartridge 410), or in any other suitable location in mechanical communication with the suture pin cartridge 410. In another implementation, a similar pawl and ratchet mechanism is coupled with the release structure 112 (as opposed to the trigger structure 110), such that a releasing action of the release structure 112 also engages the pawl and ratchet assembly to advance the suture pin cartridge 410. Other automated implementations of the suture pin advancement structures 415 use gears, cams, levers, electromagnets, servomotors, and/or any suitable mechanical, or electromechanical components to automatically advance the suture pin cartridge 410. Other suture pin advancement structures 415 can be partially or fully manual. For example, the suture pin cartridge 410 can partially protrude from the housing of the automated laparoscopic closure device 100 (e.g., and/or include one or more manually accessible structural features) by which a human operator can manually rotate the suture pin cartridge 410 to position a next suture pin 116 in the load position. Such implementations can include additional features to provide visual, tactile, or other feedback to indicate to the human user that the cartridge is advanced by a desired amount.

Other embodiments can implement the suture pin cartridge 410 in other ways. Some embodiments include suture pin cartridges 410 to support more or fewer suture pins 116 than what is illustrated. Some embodiments implement a stacked-type of cartridge having the suture pins 116 stacked one on top of the next, each being pushed into a loading place in turn by suture pin advancement structures (e.g., such as the block 126 and spring 124 arrangement illustrated in FIG. 1B). Some embodiments of the automated laparoscopic closure device 100 include internal storage for one or more additional suture pin cartridges 410 to be installed as needed. Some embodiments of the automated laparoscopic closure device 100 housing include features (e.g., a slot) to permit easy removal of a spent suture pin cartridge 410 and installation of a new suture pin cartridge 410. In some such embodiments, the suture pins 116 can be loaded one pair at a time.

The above embodiments illustrate the automated laparoscopic closure device 100 as having the ASC assembly 145 is disposed “above” the material capture region 108 and the containment sub-assembly 140 disposed “below” the material capture region 108. In such a configuration, the suture pins 116 are pushed downward through the material around a defect, and the resulting stitch forms with the knot at the underside of the defect (e.g., inside the patient), such as shown in FIG. 2E. In other embodiments, the automated laparoscopic closure device 100 is configured with the ASC assembly 145 is disposed below the material capture region 108 (in the defect insertion region 106) and the containment sub-assembly 140 disposed above the material capture region 108. In such a configuration, the suture pins 116 are pulled or pushed upward through the material around a defect, and the resulting stitch forms with the knot at the top of the defect (e.g., outside the patient). In one such embodiment, the pin deployment structure 114 of the AFR assembly 135 includes a lever structure disposed in the defect insertion region 106 below the ASC assembly 145 that converts downward motion of the trigger structure 110 into upward force on a suture pin 116 in a load position (e.g., the point of the suture pin 116 pointing upward toward the inside layer of material captured in the material capture region 108). Similar to embodiments described above, when the suture pin 116 is pushed upward through the material, it can be pushed into a pin capture structure 122 located at the top of the material capture region 108, and can subsequently pushed by a next suture pin 116 into a pin container structure 120 located above the material capture region 108 or in any suitable location within the automated laparoscopic closure device 100. In another such embodiment, the pin deployment structure 114 is configured to pierce through the material captured in the material capture region 108 and to engage a suture pin 116 chambered in a load position on the inside of the material, and to pull the suture pin 116 up through the material. For example, depressing the trigger structure 110 causes the pin deployment structure 114 to pierce through the material captured in the material capture region 108 and to engage with a suture pin 116 (e.g., by friction fitting inside or around a feature of the suture pin 116, by strong magnetic coupling with the suture pin, and/or in any other suitable manner). Releasing pressure from the trigger structure 110 can cause the release structure 112 to withdraw the pin deployment structure 114 through the material, pulling the suture pin 116 and its coupled suture 220 through the material. Another structural feature in the release path above the material capture region 108 can decouple the suture pin 116 from the pin deployment structure 114, so that the suture pin 116 remains in a pin capture structure 122 or a pin container structure 120 to hold the coupled end of the suture 220 in place. Embodiments configured to push or pull the suture pins 116 upward through the material can further be configured to accommodate suture pin cartridges 410 and/or other features.

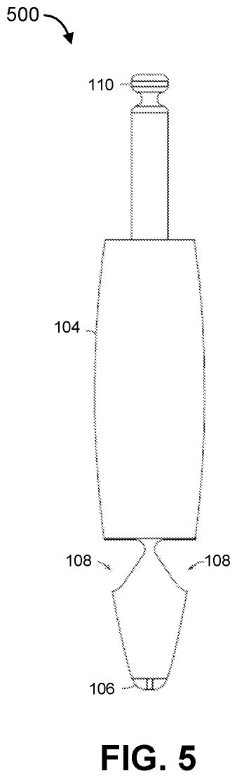

FIG. 5 shows a front view of another embodiment of an automated laparoscopic closure device 100 having a two-sided material capture region 108. Such an embodiment can be designed to push suture pins 116 concurrently into material on opposite of a defect, for example, with a single activation of the trigger structure 110. The AFR region 104 can be designed, such that the pin deployment structure 114 includes multiple firing rods, all coupled with the trigger structure 110; and suture pins 116 chambered in load positions on either side of the defect. Depressing the trigger structure 110 causes the multiple firing rods concurrently to travel downward, concurrently engaging the multiple suture pins 116, and concurrently pushing the multiple suture pins 116 through different portions of the material and into respective portions of a containment sub-assembly 140.

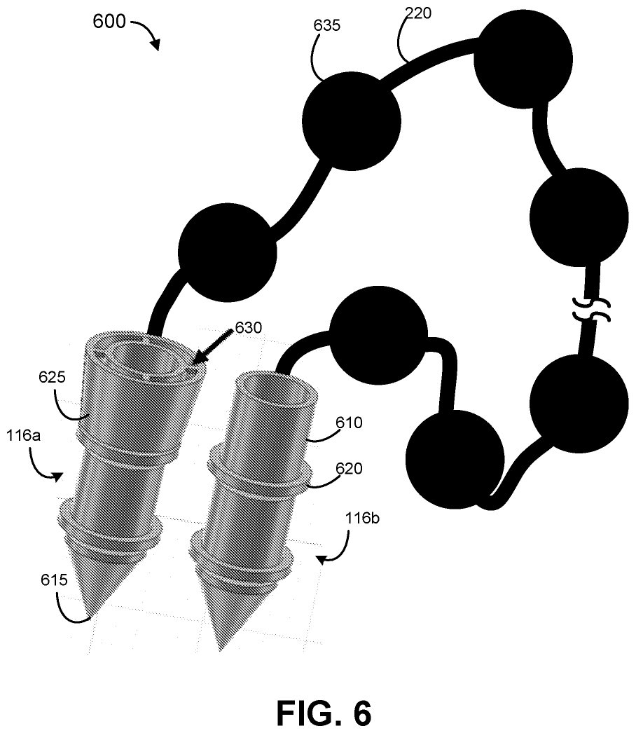

The descriptions above describe pushing or pulling suture pins 116 through material surrounding a defect. FIG. 6 shows an example of a pair of suture pins 116 coupled together by a suture 220 to illustrate features of various embodiments described herein. The illustrated suture pins 116 include a first suture pin 116a and a second suture pin 116b. Each suture pin 116 can have a main body 610 that terminates at one end in a point 615 to facilitate passage through the material. On the illustrated implementation, each suture pin 116 has a substantially cylindrical main body 610 with a pointed end 615. In another implementation, each suture pin 116 has a tapered main body 610 ending in a point 615. Any other suitable shape can be used for the suture pins 116. Some embodiments further include one or more structural features 620 integrated with the main body, such as rings, divots, channels, ribs, etc. Such features can provide added strength to the structure of the suture pins 116, can further assist with passage of the suture pins 116 through the material, can help the suture pins 116 to securely interface with the pin capture structure 122, and/or can provide any other suitable functions. The pair of suture pins 116 can be coupled together in any respective location by the suture 220. For example, each end of the suture 220 can be coupled at or near the top of the main body 120 of a respective one of the suture pins 116. Some implementations can couple together the suture pins 116 using multiple sutures 220 and/or using additional structural features (e.g., channels, notches, adhesive etc.) to facilitate the coupling of the suture(s) 120 to the suture pins 116.

In some embodiments, one of the suture pins 116 of the pair includes a ratchet-locking structure 625. The ratchet-locking structure 625 is coupled in a break-away fashion with the main body 610 of the suture pin 116. For example, the ratchet-locking structure 625 is friction fit into place, or is coupled with break-away tabs 630, or the like. In such embodiments, the suture 220 is coupled at one end to the ratchet-locking structure 625 (rather than being coupled with the main body 610 of the suture pin 116), and the suture 220 includes beads 635. The ratchet-locking structure 625 and the beads 635 are sized and shaped, such that the beads 635 can be pulled through the ratchet-locking structure 625 in one direction once the ratchet-locking structure 625 is separated from the suture pin 116, but the beads 635 are prevented from passing through the ratchet-locking structure 625 in the other direction. For example, the ratchet-locking structure 625 can include an inner taper that begins at an inner diameter slightly larger than the diameter of the beads 635 and ends at an inner diameter less than the diameter of the beads. The beads 635 can be slightly compressible while being pulled through the inner taper, such that the beads 635 can be pulled through the slightly smaller inner diameter at the end of the taper and become effectively locked at the other side of the ratchet-locking structure 625. The beads 635 can be any suitable shape and size for being pulled relatively easily through the ratchet-locking structure 625, while being relatively difficult to pull back through the ratchet-locking structure 625 in the opposite direction. For example, the beads 635 can be substantially spherical, conical, tooth-shaped, etc. In some embodiments, the beads 635 are shaped without any sharp edges, or the like, to facilitate smooth passage through the material, to avoid catching on or tearing the material, etc.

For the sake of illustration, the first suture pin 116a includes the ratchet-locking structure 625, and the second suture pin 116b does not. As described with reference to FIGS. 2A and 2B, depressing the trigger structure 110 a first time causes the pin deployment structure 114 to travel in the direction of, and to engage with, the first of the suture pins 116a. Further depressing the trigger structure 110 pushes the first suture pin 116a from its chambered load position, through first material 110a captured in the material capture region 108, and into the pin capture structure 122 of the containment sub-assembly 140. Referring again to FIG. 2C, depressing the trigger structure 110 a second time (after rotating the automated laparoscopic closure device 100 to capture second material in the material capture region 108) causes the pin deployment structure 114 to travel in the direction of, and to engage with, the second of the suture pins 116b. Further depressing the trigger structure 110 pushes the second suture pin 116b from its chambered load position, through the second material 110b now captured in the material capture region 108, and into the pin capture structure 122 of the containment sub-assembly 140.

The second suture pin 116b (e.g., the main body 610) is designed to pass through and break away the ratchet-locking structure 625 from the first suture pin 116a. For example, the pin capture structure 122 is designed to allow the main body 610 of the first suture pin 116a to be pushed by the second suture pin 116b into the pin container structure 120, but forces the ratchet-locking structure 625 of the first suture pin 116a to break away and be left behind (i.e., not in the pin container structure 120). Now, the two suture pins 116 are connected by the suture 220, and the suture pins 116 are both being held by the containment sub-assembly 140 (the first in the pin container structure 120, and the second in the pin capture structure 122). As illustrated, the suture 220 is coupled at a second end to the main body 610 of the second suture pin 116b and at a first end to the ratchet-locking structure 625 of the first suture pin 116a.

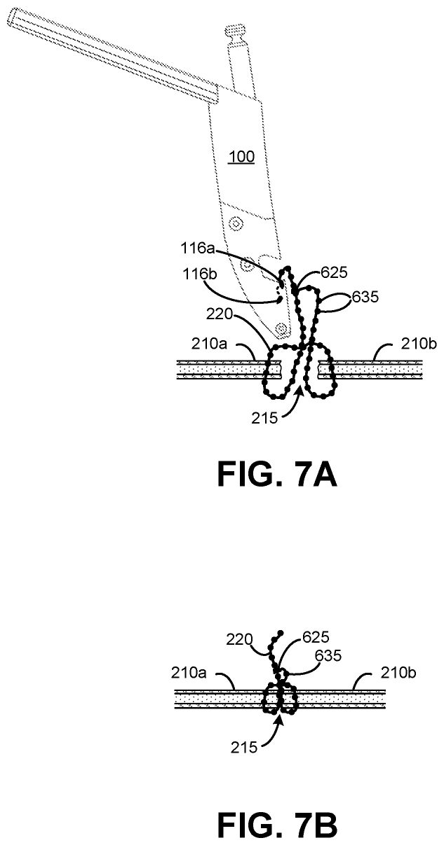

FIG. 7A shows an example of the automated laparoscopic closure device 100 in context of a defect 215 in material 210 in a position similar to that of FIG. 2E, but with a self-locking suture configuration. It can be seen that the ratchet-locking structure 625 is broken away from the main body 610 of the first suture pin 116a, and only the second end of the suture 220 is being held by the containment sub-assembly 140 of the automated laparoscopic closure device 100 (the main body of the suture pin 610 is broken away from the ratchet-locking structure 625 and is being held in the pin container structure 120). After the automated laparoscopic closure device 100 is removed and pulled away from the defect, the second side of the suture 220 can be used to successively pull beads 635 through the ratchet-locking structure 625, successively tightening the loop of the suture 220 and drawing together the sides of the defect to close the defect. For example, FIG. 7B shows an example of the self-locking suture having been cut away from the automated laparoscopic closure device 100 and having been partially cinched to close the defect 215 in the material 210. The illustrated scenario of FIG. 7B is similar to that of FIG. 2F, but with the self-locking suture. When the suture 220 is cinched to an appropriate tightness, the loop of the suture 220 is effectively locked in size (i.e., similar to being tied off) by one of the beads 635 and the ratchet-locking structure 625. The excess portion of the suture 220 can be trimmed to leave behind a completed stitch.

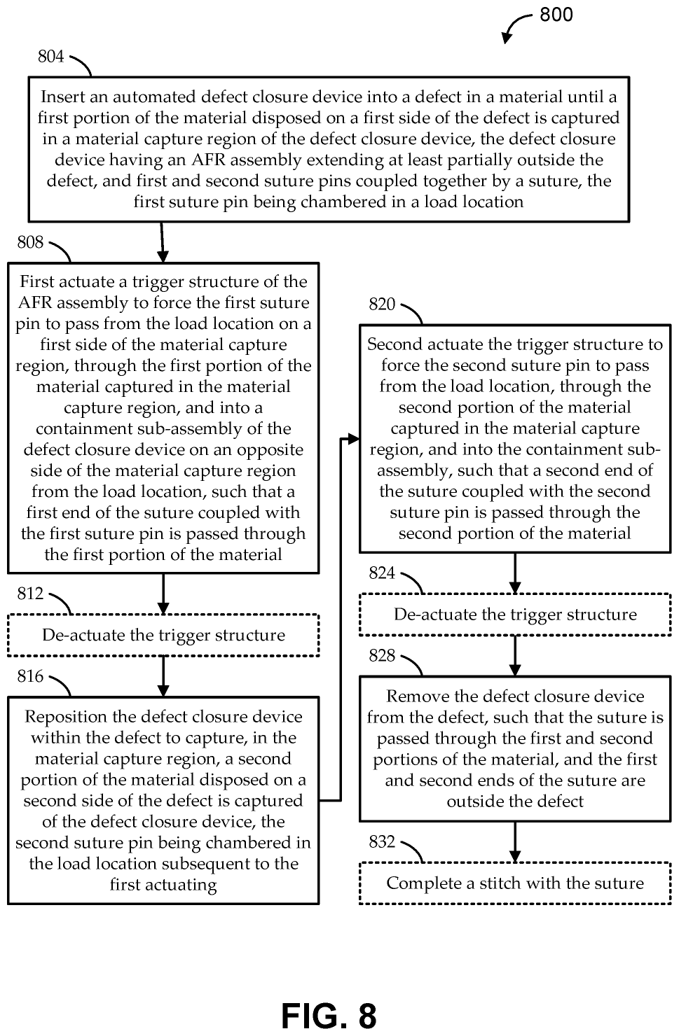

FIG. 8 shows a flow diagram of an illustrative method 800 for closure of a defect in material, according to various embodiments described herein. Embodiments of the method 800 can be implemented using any of the device embodiments described herein, and/or any other suitable devices or systems. Embodiments begin at stage 804 by inserting an automated defect closure device into a defect in a material until a first portion of the material disposed on a first side of the defect is captured in a material capture region of the defect closure device. As described herein, the defect closure device can have an automated fire and release (AFR) assembly extending at least partially outside the defect, and first and second suture pins coupled together by a suture, the first suture pin being chambered in a load location. At stage 808, embodiments can first actuate a trigger structure of the AFR assembly to force the first suture pin to pass from the load location on a first side of the material capture region, through the first portion of the material captured in the material capture region, and into a containment sub-assembly of the defect closure device on an opposite side of the material capture region from the load location. This can result in a first end of the suture coupled with the first suture pin being passed through the first portion of the material.

At stage 816, embodiments can reposition the defect closure device within the defect to capture, in the material capture region, a second portion of the material disposed on a second side of the defect is captured of the defect closure device. For example, the repositioning can involve rotating the defect closure device substantially 180 degrees, such that the second portion may be substantially across from the first portion (i.e., on the opposite side of the defect). In some embodiments, prior to the repositioning at stage 816, the trigger structure can be de-actuated at stage 812. The de-actuating can cause the trigger structure and/or a pin deployment structure to be returned (e.g., by a release structure) to a reset position. In some implementations, the de-actuating at stage 812 further causes the second suture pin to become chambered in the load location. In other implementations, the second suture pin is chambered in the load location subsequent to the first actuating at stage 808 in any suitable manner.

At stage 820, embodiments can second actuate the trigger structure to force the second suture pin to pass from the load location, through the second portion of the material captured in the material capture region, and into the containment sub-assembly, such that a second end of the suture coupled with the second suture pin is passed through the second portion of the material. At stage 828, embodiments can remove the defect closure device from the defect, such that the suture is passed through the first and second portions of the material, and the first and second ends of the suture are outside the defect. In some embodiments (e.g., prior or subsequent to the removing at stage 828), the trigger structure can be second de-actuated at stage 824. In some embodiments, the defect closure device includes multiple pairs of suture pins (e.g., in a specially designed suture pin cartridge), and each pair is coupled together by a respective suture. In such embodiments, subsequent to the second actuating at stage 820, the second de-actuating at stage 824 can cause chambering of a third suture pin in the load position, where the third suture pin is coupled with a fourth suture pin by a second suture (i.e., the first and second suture pins constitute a first pair, and the third and fourth suture pins constitute a second pair).

Some embodiments, at stage 832, can complete a stitch with the inserted ends of the suture. Some such embodiments, subsequent to the removing at stage 828, can cinch the first and second ends of the suture to pull together the first and second portions of the material. The ends of the suture can be cut and tied together to complete the stitch. Other such embodiments can use a self-locking suture configuration. For example, the suture includes beads spaced along a length of the suture (e.g., integrated into the material of the suture). The first suture pin includes a ratchet-locking structure removably coupled with a main body, the first end of the suture coupled with the ratchet-locking structure, and the ratchet-locking structure is configured to permit passage of the beads in a tightening direction and to restrict passage of the beads in a loosening direction opposite the tightening direction. The second actuating at stage 820 can force the second suture pin into the containment sub-assembly through the ratchet-locking structure of the first suture pin, thereby de-coupling the ratchet-locking structure from the main body of the first suture pin. In such an embodiment, at stage 832, completing the stitch can involve successively pulling each of a portion of the beads through the ratchet-locking structure in the tightening direction to cinch and lock together the first and second portions of the material, thereby forming the stitch.

It will be understood that, when an element or component is referred to herein as “connected to” or “coupled to” another element or component, it can be connected or coupled to the other element or component, or intervening elements or components may also be present. in contrast, when an element or component is referred to as being “directly connected to,” or “directly coupled to” another element or component, there are no intervening elements or components present between them. It will be understood that, although the terms “first,” “second,” “third,” etc. may be used herein to describe various elements, components, these elements, components, regions, should not be limited by these terms. These terms are only used. to distinguish one element, component, from another element, component. Thus, a first element, component, discussed below could be termed a second element, component, without departing from the teachings of the present invention.

As used herein, the terms “a”, “an” and “the” may include singular and plural references. it will be further understood that the terms “comprising”, “including”, having” and variants thereof, when used in this specification, specify the presence of stated features, steps, operations, elements, and/or components, but do not preclude the presence or addition of one or more other features, steps, operations, elements, components, and/or groups thereof. In contrast, the term “consisting of” when used in this specification, specifies the stated features, steps, operations, elements, and/or components, and precludes additional features, steps, operations, elements and/or components. Furthermore, as used herein, the words “and/or” may refer to and encompass any possible combinations of one or more of the associated listed items.

While the present invention is described herein with reference to illustrative embodiments, this description is not intended to be construed in a limiting sense. Rather, the purpose of the illustrative embodiments is to make the spirit of the present invention be better understood by those skilled in the art. In order not to obscure the scope of the invention, many details of well-known processes and manufacturing techniques are omitted. Various modifications of the illustrative embodiments, as well as other embodiments, will be apparent to those of skill in the art upon reference to the description. It is therefore intended that the appended claims encompass any such modifications. Furthermore, some of the features of the preferred embodiments of the present invention could be used to advantage without the corresponding use of other features. As such, the foregoing description should be considered as merely illustrative of the principles of the invention, and not in limitation thereof. Those of skill in the art will appreciate variations of the above-described embodiments that fall within the scope of the invention. As a result, the invention is not limited to the specific embodiments and illustrations discussed above, but by the following claims and their equivalents.

What is claimed is:

1. A method for closure of a defect in material comprising:
inserting an automated defect closure device into a defect in a material until a first portion of the material disposed on a first side of the defect is captured in a material capture region of the defect closure device, the defect closure device having an automated fire and release (AFR) assembly extending at least partially outside the defect, and first and second suture pins coupled together by a suture, the first suture pin being chambered in a load location;
first actuating a trigger structure of the AFR assembly to force the first suture pin to pass from the load location on a first side of the material capture region, through the first portion of the material captured in the material capture region, and into a containment sub-assembly of the defect closure device on an opposite side of the material capture region from the load location, such that a first end of the suture coupled with the first suture pin is passed through the first portion of the material;
repositioning the defect closure device within the defect to capture, in the material capture region, a second portion of the material disposed on a second side of the defect is captured of the defect closure device, the second suture pin being chambered in the load location subsequent to the first actuating;
second actuating the trigger structure to force the second suture pin to pass from the load location, through the second portion of the material captured in the material capture region, and into the containment sub-assembly, such that a second end of the suture coupled with the second suture pin is passed through the second portion of the material; and
removing the defect closure device from the defect, such that the suture is passed through the first and second portions of the material, and the first and second ends of the suture are outside the defect.

2. The method of claim 1, further comprising:
de-actuating, subsequent to the first actuating, the trigger structure to cause a release structure to return the trigger structure to a reset position and to cause a suture pin advancement structure to chamber the second suture pin in the load position.

3. The method of claim 2, wherein:
the first and second suture pins are in a suture pin cartridge coupled with the suture pin advancement structure; and
the de-actuating causes the suture pin advancement structure to reposition the suture pin cartridge so as to chamber the second suture pin in the load position.

4. The method of claim 1, wherein the defect closure device comprises a plurality of pairs of suture pins, each of the plurality of pairs of suture pins being coupled together by a respective suture, the first and second suture pins being a first of the plurality of pairs of suture pins, and further comprising:
de-actuating, subsequent to the second actuating, the trigger structure to cause a release structure to return the trigger structure to a reset position and to cause a suture pin advancement structure to chamber a third suture pin in the load position, the third suture pin and a fourth suture pin being a second of the plurality of pairs of suture pins.

5. The method of claim 1, wherein the repositioning comprises rotating the defect closure device within the defect, such that the second portion of the material is across the defect from the first portion of the material.

6. The method of claim 1, further comprising:
subsequent to the removing, cinching the first and second ends of the suture to pull together the first and second portions of the material, and cutting and tying together the first and second ends of the suture to form a stitch.

7. The method of claim 1, wherein:
the first actuating comprises depressing the trigger structure to cause a pin deployment structure to physically engage with the first suture pin in the load position and to force the first suture pin to pass through the first portion of the material captured and into the containment sub-assembly; and
the second actuating comprises depressing the trigger structure to cause the pin deployment structure to physically engage with the second suture pin in the load position and to force the second suture pin to pass through the second portion of the material captured and into the containment sub-assembly.

8. The method of claim 7, wherein:
the first actuating forces the first suture pin into a pin capture structure of the containment sub-assembly; and
the second actuating forces the second suture pin into the pin capture structure of the containment sub-assembly, thereby forcing the first suture pin out of the pin capture structure and into a pin container structure.

9. The method of claim 7, wherein:
the suture comprises a plurality of beads spaced along a length of the suture;
the first suture pin comprises a ratchet-locking structure removably coupled with a main body, the first end of the suture coupled with the ratchet-locking structure, the ratchet-locking structure configured to permit passage of the plurality of beads in a tightening direction and to restrict passage of the plurality of beads in a loosening direction opposite the tightening direction; and
the second actuating forces the second suture pin into the containment sub-assembly through the ratchet-locking structure of the first suture pin, thereby de-coupling the ratchet-locking structure from the main body of the first suture pin.

10. The method of claim 9, further comprising:
subsequent to the removing, successively pulling each of a portion of the plurality of beads through the ratchet-locking structure in the tightening direction to cinch and lock together the first and second portions of the material, thereby forming a stitch.

11. An automated defect closure device comprising:
a defect insertion region configured to be inserted into a defect in a material;
a material capture region configured to capture a portion of the material adjacent to the defect when the defect insertion region is inserted into the defect;
an automated fire and release (AFR) region to house a AFR assembly, wherein the material capture region is disposed between the defect insertion region and the AFR region;
an automated suture cartridge (ASC) assembly to house a first suture pin and a second suture pin, each coupled to a respective end of a suture; and
a containment sub-assembly,
wherein the AFR assembly is configured, upon activation, to force one of the suture pins to pass from a load location of the ASC assembly through the portion of the material captured in the material capture region and into the containment sub-assembly, such that the respective end of the suture coupled with the one of the suture pins is passed through the portion of the material.

12. The automated defect closure device of claim 11, further comprising:
a body portion comprising the defect insertion region, the material capture region and the AFR region; and
a handle portion coupled with the body portion at a coupling region,
wherein the handle portion extends from the coupling region generally in a first direction defining a first axis, the body portion extends from the coupling region generally in a second direction defining a second axis, and the first and second axes form an obtuse angle.

13. The automated defect closure device of claim 11, wherein the AFR assembly is configured:
upon first activation, to force the first suture pin to pass from the load location of the ASC assembly through a first portion of the material captured in the material capture region and into the containment sub-assembly, such that the first end of the suture is passed through the first portion of the material; and
upon second activation, subsequent to the automated defect closure device being rotated within the defect to capture, in the material capture region, a second portion of the material adjacent to the defect apart from the first portion of the material, to force the second suture pin to pass from the load location of the ASC assembly through the second portion of the material and into the containment sub-assembly, such that the second end of the suture is passed through the second portion of the material.

14. The automated defect closure device of claim 13, wherein the AFR assembly is configured:
upon the first activation, to force the first suture pin into a pin capture structure of the containment sub-assembly; and
upon the second activation, to force the second suture pin into the pin capture structure, thereby forcing the first suture pin out of the pin capture structure and into a pin container structure of the containment sub-assembly.

15. The automated defect closure device of claim 11, wherein:
the AFR assembly comprises a trigger structure, a pin deployment structure coupled with the trigger structure; and
the AFR assembly is configured, upon activation of the trigger structure, to engage the pin deployment structure with the one of the suture pins in the load location of the ASC assembly, such that the pin deployment structure forces the one of the suture pins from the load location, through the portion of the material, and into the containment sub-assembly.

16. The automated defect closure device of claim 15, wherein:
the AFR assembly further comprises a release structure configured, upon de-activation of the trigger structure, to:
disengage the pin deployment structure from the one of the suture pins, thereby leaving the one of the suture pins contained by the containment sub-assembly; and
return the trigger structure and the pin deployment structure at least partially to a reset position.

17. The automated defect closure device of claim 16, wherein:
the trigger structure comprises a plunger structure that is activated by depressing;
the pin deployment structure comprises a pushing rod; and
the release structure comprises a spring.

18. The automated defect closure device of claim 16, wherein:
the release structure is further configured, upon de-activation of the trigger structure, automatically to chamber the second suture pin in the load location of the ASC assembly.

19. The automated defect closure device of claim 11, wherein the ASC assembly comprises:
a suture pin cartridge configured to hold a plurality of suture pins including the first and second suture pins; and
a suture pin advancement structure configured to interface with the suture pin cartridge to selectively advance each of the plurality of suture pins into the load position.

20. The automated defect closure device of claim 19, wherein the suture pin cartridge is configured to hold a plurality of pairs of suture pins, each pair of suture pins having a respective first and second suture pins coupled together by a respective suture.
